(19) United States
(12) Patent Application Publication
(10) Pub. No.: US 2024/0122820 A1
(43) Pub. Date: Apr. 18, 2024

(54) Aerosol Personal Care Products

(71) Applicant: The Procter & Gamble Company, Cincinnati, OH (US)

(72) Inventors: Elton Luis Menon, Mason, OH (US); Julie Beth Hipp, Cincinnati, OH (US); Matthew John Martin, California, KY (US); Ke Ming Quan, West Chester, OH (US); Julie Savchenko, Maineville, OH (US); David Frederick Swaile, Cincinnati, OH (US)

(21) Appl. No.: 18/488,521

(22) Filed: Oct. 17, 2023

Related U.S. Application Data
(60) Provisional application No. 63/416,622, filed on Oct. 17, 2022.
(60) Provisional application No. 63/416,623, filed on Oct. 17, 2022.
(60) Provisional application No. 63/416,624, filed on Oct. 17, 2022.

(51) Int. Cl. A61K 8/04; A45D 34/04; A61Q 15/00; B65D 83/14

(52) U.S. Cl. CPC A61K 8/046; A45D 34/04; A61Q 15/00; B65D 83/14

(57) ABSTRACT

An aerosol personal care product comprising a composition comprising a compressed gas propellant and a single-phase liquid concentrate; wherein the concentrate comprises at least about 10%, by weight of the concentrate, of one or more emollients and wherein at least one emollient has a viscosity of at least about 20 cP; and wherein the concentrate has a viscosity (cP) to surface tension (dyn/cm) ratio of at most about 1.

### TECHNICAL FIELD

One aspect of the invention relates generally to aerosol personal care products.

### BACKGROUND OF THE INVENTION

Spray devices are generally well known in the art, examples of which are disclosed in U.S. Pat. Nos. 4,396,152 and 5,082,652. Aerosol products are broadly used in a variety of applications for the beauty industry including antiperspirants, deodorants, hair sprays, and body lotions. Aerosol products traditionally use liquefied propellants which change from a liquid to a gaseous phase at critical temperatures and pressures. Examples of liquefied propellants are hydrocarbons and hydrofluorocarbons.

However, liquified propellants have two major concern areas: Liquified propellants are flammable, with the possibility of explosion if the propellant to air ratio exceeds explosive limits. Thus, liquefied propellants are in general considered dangerous goods requiring, in many geographies, regulated/specialized handling, transportation, and storage. The other disadvantage of these propellants is their environmental impact. For example, hydrofluorocarbons are a green-house gas and hydrocarbons are volatile organic compounds that form ozone/smog (T. J. Wallington et al. /Chemosphere 129 (2015) 135-141). Thus, compressed gases, which are in the gas state at 25° C. and at a pressure of at least 50 psi, such as nitrogen, air, carbon dioxide, are a safer and more environmentally compatible alternative to liquefied propellants.

Aerosol systems that use compressed gases are known in the art, for example, U.S. Pat. No. 11,059,659 and U.S. Patent Publication No. 20220143629. In general, compressed gas products use propellants that are in the gas phase at 25° C. and at pressures of at least 50 psi. Upon actuation, the gas pushes the product through the valve and actuator where the product meets the outlet nozzle or actuator insert and atomization takes place.

Surprisingly, the present inventors found that products using compressed gas were very sensitive to the product viscosity, with most examples not able to spray liquids with viscosity of about 20 cP (0.02 Pa*s) and above. This difficulty spraying higher viscosity products was found to be true even for effervescent atomization spray devices, such as those described in U.S. Pat. No. 11,059,659, which work by creating a two phase “bubble-laden (“bubbly”)” product stream that is supplied to the nozzle, thus enhancing atomization. Ingredients and formulated products with high viscosity are broadly used in the beauty spray industry, providing a myriad of benefits to consumers. Without intending to be bound by any theory, it is believed that for products meant to be sprayed onto consumers (e.g., hair sprays, deodorants, lotions) a low viscosity formula could be perceived as runny or wet. This can be particularly undesirable, especially for products applied on axilla, such as deodorants, in which a dry application connotes protection against odor and wetness.

Spraying viscous formulations and ingredients using compressed gases thus become an important proposition.

### SUMMARY OF THE DISCLOSURE

An aerosol personal care product comprising a composition, wherein the composition comprises:
a) a compressed gas propellant; and
b) a single-phase liquid concentrate, wherein the concentrate comprises at least about 10%, by weight of the concentrate, of one or more emollients and wherein at least one emollient has a viscosity of at least about 20 cP;
and wherein the concentrate has a viscosity (cP) to surface tension (dyn/cm) ratio of at most about 1.

### BRIEF DESCRIPTION OF THE DRAWINGS

While the specification concludes with claims, it is believed that the same will be better understood from the following description taken in conjunction with the accompanying drawings wherein like numbers illustrate like elements throughout the views and in which:

FIG. 1 is a cross-sectional side view of one non-limiting example of a novel spray device comprising an actuator, a valve assembly and a reservoir containing a compressed gas propellant and a composition;

FIG. 2 is a perspective view of the valve assembly of FIG. 1;

FIG. 3 is a side elevation view of the valve assembly of FIG. 2;

FIG. 4 is a cross-sectional view of the valve assembly of FIG. 3, taken along 5-5 thereof;

FIG. 5 is cross-sectional side elevation view of the valve stem of FIG. 4;

FIG. 6 is a perspective view of the seal of FIG. 4;

FIG. 7 is a perspective view of the housing of FIG. 4;

FIG. 8 is a cross-sectional side elevation view of the housing of FIG. 7, taken along line 9-9 thereof;

FIG. 9 is a perspective view of the insert of FIG. 4;

FIG. 10 is a cross-sectional side elevation view of the insert of FIG. 9, taken along line 11-11 thereof; and

FIG. 11 is a bottom plan view of the insert of FIG. 9.

### DETAILED DESCRIPTION

A device, container, composition, blowing agent, etc. may comprise, consist essentially of, or consist of, various combinations of the materials, features, structures, and/or characteristics described herein.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments of the present invention, all percentages are by weight of the antiperspirant or deodorant composition (or formulation), unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. The term “molecular weight” or “M.Wt.” as used herein refers to the number average molecular weight unless otherwise stated.

The term “antiperspirant composition” refers to any composition containing an antiperspirant active and which is intended to be applied onto skin. The term “deodorant composition” refers to any composition containing a deodorant active and which is intended to be applied onto skin.

The term “at the time of making” refers to a characteristic (e.g., viscosity) of a raw material ingredient just prior to mixing with other ingredients.

The term “container” and “device” and derivatives thereof refers to the package that is intended to store and dispense an antiperspirant or deodorant composition. A container or device may typically comprise a reservoir for storing the antiperspirant or deodorant composition, a valve for controlling flow of the antiperspirant or deodorant composition, and an actuator by which a user can actuate the valve.

The term “substantially free of” refers to an amount of a material that is less than 1%, 0.5%, 0.25%, 0.1%, 0.05%, 0.01%, or 0.001% by weight of an antiperspirant composition. “Free of” refers to no detectable amount of the stated ingredient or thing.

The term “total fill” or “total fill of materials” refers to the total amount of materials added to or stored within a reservoir(s) of a container. For example, total fill includes the blowing agent and antiperspirant or deodorant composition stored within a device after completion of filling and prior to first use.

The term “viscosity” means dynamic viscosity (measured in centipoise, cPs, or Pascal-second, Pa·s) or kinematic viscosity (measured in centistokes, cst, or m2/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or another substitutable model known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

The Term Dv50 Spray particle refers to the average particle size of spray droplets and test results were obtained by the method described in U.S. Patent Publication No. 2015/0000687. A Malvern Spraytec instrument was used to measure the particle size distribution following the manufacturer's instructions with test samples having a temperature between 20° C. to 22° C. and samples were sprayed perpendicular to the laser bean for 5 seconds. The lower the Dv50 the finer the aerosol. A finer aerosol indicates easier atomization of the formula and vice versa, the higher the Dv50 the harder to atomize up to the point where no aerosol is formed and there will be no results for Dv50, or at least inconsistent spray quality.

The term surface tension refers to the tension of the surface film of a liquid caused by the attraction of the particles in the surface layer by the bulk of the liquid, which tends to minimize surface area. Surface tension measurements were done using Wilhelmy plate method ASTM D1331-20 method C, with a Krüss K100 tensiometer instrument used for all measurements.

As discussed, spraying viscous formulations and ingredients using compressed gases is an important proposition. To this aim, many publications in the literature indicate that to improve atomization, reduction of surface tension is one of the most common approaches (C. E. Ejim et al Fuel 89 (2010) 1872-1882) (Aniket P. Kulkarni et al International Journal of Multiphase Flow 133 (2020) 103448). Surprisingly, the present inventors have found that for compressed gas systems, the opposite is true. The present inventors believe that given a certain viscosity, increasing the surface tension (dyn/cm) to above the formulation viscosity (cP) can create the conditions that are conducive for atomization upon spraying. In general, the lower the ratio of viscosity to surface the easier the atomization, as reflected by achieving a lower median particle size of the aerosol.

Tables 1-5 below illustrate that the ratio of a composition's viscosity to its surface tension impacts the ability for the composition to be effectively sprayed via effervescent atomization.

Table 1 below shows Examples 1-10 that were prepared by adding the ingredients into a suitable container and mixing them until all ingredients were homogeneous. Examples 1-5, 7, 8, and 10 are a mixture, or concentrate, that could then have a compressed gas propellant added to make inventive composition. Examples 6 and 9 are mixtures, or concentrates, of materials that serve as Comparative Examples to the concentrates of inventive compositions. Table 2 shows the viscosity (cP) and surface tension (dyn/cm) for each of Examples 1-10, along with the ratio of the viscosity to surface tension. The viscosity measurements were made at 21° C. using a Brookfield RVT Viscometer Model employing an RV-01 spindle at 20 rpm and techniques well known in the art. Surface tension measurements were done using Wilhelmy plate method ASTM D1331-20 method C, with a Krüss K100 tensiometer instrument used for all measurements.

In some cases, inventive products may comprise an emulsion, either an oil-in-water emulsion or a water-in-oil emulsion. In water-in-oil emulsions, the continuous phase may comprise concentrate formulations such as Examples 1-5, 7, 8, and 10.

TABLE 1
Concentration formulations

| | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Ingredients | (wt. %) | (wt. %) | (wt. %) | (wt. %) | (wt. %) | (wt. %) | (wt. %) | (wt. %) | (wt. %) | (wt. %) |
| Ethanol 200 | 67.84 | 49.04 | 30.24 | 67.84 | 32.16 | | | | | |
| proof | | | | | | | | | | |
| Dipropylene | 18.80 | 37.60 | 56.41 | | | | 70.00 | 30.00 | 100.00 | |
| glycol | | | | | | | | | | |
| Caprilic/capric | 9.90 | 9.90 | 9.90 | 32.16 | 67.84 | 100.00 | | | | |
| triglyceride | | | | | | | | | | |
| Propylene | | | | | | | 30.00 | 70.00 | | 100.00 |
| Carbonate | | | | | | | | | | |
| Water | 3.46 | 3.46 | 3.46 | | | | | | | |

TABLE 2
viscosity and surface tension

| | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Surface tension | 24.4 | 26.1 | 28.2 | 23.0 | 23.8 | 26.3 | 34.9 | 34.7 | 33.3 | 40.8 |
| (dyn/cm) | | | | | | | | | | |
| Viscosity (cP) | 7.5 | 10.0 | 16.3 | 6.3 | 12.5 | 31.3 | 30.0 | 12.5 | 122.1 | 10.0 |
| Viscosity/surface | 0.31 | 0.38 | 0.58 | 0.27 | 0.52 | 1.19 | 0.86 | 0.36 | 3.67 | 0.24 |
| tension | | | | | | | | | | |

Unlike liquefied propellants the % w/w amount of compressed gas in the final composition depends on how much empty space is left in the can after the can is filled with the formula concentrate.

Table 3 shows Examples 11-20, which are each 100 g of formulations of example 1-10. Each of Examples 1-10 were placed into a 45 mm×150 mm aluminum aerosol can, which was then fitted with a valve, crimped, and pressurized with nitrogen to 100±5 psi. Samples were prepared in such a way that, for example, example 11 used 100 g of example 1 formulation and example 12 used 100 g of example 2 formulation and so on. Examples 11-20 used Coster® spray system COSTERECO™ using a KV valve with no vapor tap and one stem orifice of 0.010 inch and a tail orifice of 0.042 inch. A Coster® actuator with a 0.010 inch mechanical breakdown insert and a stem volume reducer center post that fits into the valve stem ID.

The particle size distribution for each of Examples 11-20 is shown in Table 3. Spray particle size Dv50 was done as per U.S. Patent Publication No. 2015/0000687. Without intending to be bound by any theory, it is believed that a good quality spray atomization is one in which the Dv50 is at about no higher than about 200 μm while a consumer is using the product (from 2 to 10 seconds of application). As can be seen, the two comparative concentrates in Examples 6 and 9, in which the viscosity to surface tension ratio is greater than 1, had inconsistent or little spraying. The remaining inventive concentrates sprayed fine.

TABLE 3
D<50> with Coster ® valve and actuator

| | Ex. 11 | Ex. 12 | Ex. 13 | Ex. 14 | Ex. 15 | Ex. 16 | Ex. 17 | Ex. 18 | Ex. 19 | Ex. 20 |
|---|---|---|---|---|---|---|---|---|---|---|
| D<50> μm | 56.2 | 48.3 | 69.3 | 49.8 | 63.8 | 298.6* | 149.1 | 58.7 | 500** | 47.7 |

*Inconsistent spray with some squirting
**very little to no-atomization

Table 4 shows the particle size distribution for Examples 21-30, which are each 100 g of Examples 1-10 that were placed into 45 mm×150 mm aluminum aerosol can which were then fitted with a valve, crimped, and pressurized with nitrogen to 100±5 psi. Samples were prepared in such way that example 21 used 100 g of example 1 formulation and example 22 used 100 g of example 2 formulation and so on. Examples 21-30 use a Salvalco Eco-Valve® spray system consisting of an SSG-ECO valve with 2×0.020 inch stem lower stem orifice a single 0.018 inch upper stem orifice no vapor tap and 0.062 inch tail orifice. Salvalco system was fitted with Salvalco LP05 button with 0.010 inch mechanical breakdown actuator insert.

TABLE 4
D<50> with Salvalco Eco-Valve ® and Actuator

| | Ex. 21 | Ex. 22 | Ex. 23 | Ex. 24 | Ex. 25 | Ex. 26 | Ex. 27 | Ex. 28 | Ex. 29 | Ex. 30 |
|---|---|---|---|---|---|---|---|---|---|---|
| D<50> μm | 45.0 | 55.4 | 82.5 | 52.6 | 65.4 | 336.5** | 300.9** | 66.8 | 510.4** | 47.1 |

*Inconsistent spray with some squirting
**very little to no-atomization

Table 5 shows the particle size distribution for Examples 31-40, which are each 100 g of Example 1-10 that were placed into 45 mm×150 mm aluminum aerosol can which were then fitted with a valve, crimped, and pressurized with nitrogen to 100±5 psi. Samples were prepared in such way that example 31 used 100 g of example 1 formulation and example 32 used 100 g of example 2 formulation and so on. Examples 31 uses Lindal® neo Enhance Mist Technology™ (EMT) consisting of a LI98 valve with 4×0.030 inch stem orifice and 0.040 inch tail orifice with no vapor tap. The EMT actuator has a 0.013 inch mechanical breakdown insert.

TABLE 5
D<50> with Lindal ® Valve and Actuator

| | Ex. 31 | Ex. 32 | Ex. 33 | Ex. 34 | Ex. 35 | Ex. 36 | Ex. 37 | Ex. 38 | Ex. 39 | Ex. 40 |
|---|---|---|---|---|---|---|---|---|---|---|
| D<50> μm | 66.9 | 120.3 | 377.9** | 70.4 | 182.4 | 526.2** | 588.1** | 151.1 | *** | 72.1 |

*Inconsistent spray with some squirting
**very little to no-atomization
*** Did not atomized

As can be seen in Tables 3, 4, and 5, the lower the ratio of viscosity to surface tension, the lower the particle size, and a ratio above 1 led to difficulty spraying. Furthermore, the inventors realized that the mechanism that produces good atomization, that is, the viscosity being less than the surface tension, is limited by the difficulty to produce a formulation with high surface tension, because viscosity can easily exceed the maximum value of surface tension of a formulation, such as in tables 6-10. In these examples, the amount of polymers (VA/Crotonates/Vinyl Neodecanoate and Octylacrylamide/acrylates) increase viscosity up to a point until the viscosity exceeds the surface tension of the formula, at which point the sprays systems are no longer are able to atomize the product.

Example 41-43 were prepared by mixing the ingredients until the solution is homogeneous followed by a milling step using an IKA T50 ultraturex for at least 3 minutes.

TABLE 6
Effect off high viscosity/low surface tension

| | Ex. 41 | Ex. 42 | Ex. 43 |
|---|---|---|---|
| | (wt. %) | (wt. %) | (wt. %) |
| Purified Water | 4.37% | 4.37 | 4.37 |
| Ethanol | 89.93 | 85.83 | 77.63 |
| Mea Borate MPA Borate | 0.20 | 0.20 | 0.20 |
| Polysorbate 80 | 0.13 | 0.13 | 0.13 |
| Hexamethyldisiloxane | 0.08 | 0.08 | 0.08 |
| Ammonium Benzoate | 0.20 | 0.20 | 0.20 |
| Aminomethyl Propanol 95% | 0.55 | 0.55 | 0.55 |
| VA/Crotonates/Vinyl | 2.16 | 4.32 | 8.64 |
| Neodecanoate | | | |
| Octylacrylamide/acrylates | 1.94 | 3.88 | 7.76 |
| Ethylhexyl | 0.17 | 0.17 | 0.17 |
| Methoxycinnamate | | | |
| Redline BFM | 0.27 | 0.27 | 0.27 |

TABLE 7
high viscosity low surface tension examples viscosity/surface tension ratio

| | Ex. 41 | Ex. 42 | Ex. 43 |
|---|---|---|---|
| Viscosity (cP) | 6.25 | 16.25 | 30.00 |
| Surface tension (dyn/cm) | 23.00 | 23.00 | 23.00 |
| Viscosity/surface tension | 0.27 | 0.71 | 1.30 |

Examples 41 and 42 do not have at least about 10% emollients, and while Example 43 does, the ratio of viscosity to surface tension is over 1. This shows the difficulty of delivering at least 10% emollients and their consumer-desired benefits, while maintaining the viscosity to surface tension ratio below 1 so that the product will actually spray successfully. Examples 44-46 were placed into a 45 mm×150 mm aluminum aerosol can, which was then fitted with a valve, crimped, and pressurized with nitrogen to 100±5 psi. Samples were prepared in such a way that, for example, example 44 used 100 g of example 41 formulation and example 45 used 100 g of example 42 formulation and so on. Examples 44-46 used Coster® spray system COSTERECO™ using a KV valve with no vapor tap and one stem orifice of 0.010 inch and a tail orifice of 0.042 inch. A Coster® actuator with a 0.010 inch mechanical breakdown insert and a stem volume reducer center post that fits into the valve stem ID.

TABLE 8
High viscosity samples particle size data Coster Eco-Valve ® and actuator

| | Ex. 44 | Ex. 45 | Ex. 46 |
|---|---|---|---|
| Dv50 μm | 70.93 | 106.54 | *** |

*** Did not atomize

Examples 47-49 that were placed into 45 mm×150 mm aluminum aerosol can which were then fitted with a valve, crimped, and pressurized with nitrogen to 100±5 psi. Samples were prepared in such way that example 47 used 100 g of example 41 formulation and example 48 used 100 g of example 42 formulation and so on. Examples 47-49 use a Salvalco Eco-Valve® spray system consisting of an SSG-ECO valve with 2×0.020 inch stem lower stem orifice a single 0.018 inch upper stem orifice no vapor tap and 0.062 inch tail orifice. Salvalco system was fitted with Salvalco LP05 button with 0.010 inch mechanical breakdown actuator insert.

TABLE 9
High viscosity samples particle size data Salvalco Eco-Valve ® and actuator

| | Ex. 47 | Ex. 48 | Ex. 49 |
|---|---|---|---|
| Dv50 μm | 71.04 | 151.70 | *** |

*** Did not atomize

Example 50-52 that were placed into 45 mm×150 mm aluminum aerosol can which were then fitted with a valve, crimped, and pressurized with nitrogen to 100±5 psi. Samples were prepared in such way that example 50 used 100 g of example 41 formulation and example 51 used 100 g of example 42 formulation and so on. Examples 50-52 uses Lindal® neo Enhance Mist Technology™ (EMT) consisting of a LI98 valve with 4×0.030 inch stem orifice and 0.040 inch tail orifice with no vapor tap. The EMT actuator has a 0.013 inch mechanical breakdown insert.

TABLE 10
High viscosity samples particle size data Lindal ® valve and actuator

| | Ex. 50 | Ex. 51 | Ex. 52 |
|---|---|---|---|
| Dv50 μm | 97.11 | 514.4** | *** |

**very little to no atomization
*** Did not atomize

The data indicates that the lower the ratio of viscosity to surface tension, the easier for the spray system to produce an aerosol and thus the easier to atomize the formulation. This can be supported by the Dv50, which is smaller when the ratio of viscosity/surface tension is smaller.

Surprisingly, as the inventors compared results from table 3-5, it was found that there is a noticeable difference in the ability of the valves to atomize the formulations. It is believed that the difference is coming from the ratio of valve stem area to the actuator insert area, which impacts the ability of the system to atomize the formula. It can become particularly noticeable as the ratio of viscosity to surface tension approaches 1 (Table 11), with the ratio of the valve stem area to the actuator insert area being less than about 10 being the preferred ratio. Without intending to be bound by any theory, it is believed that by keeping the size of insert orifice closer to the valve stem orifice one avoids a rush of formula thru the actuator that may become “bottle necked” at the insert orifice. If the formula bottle necks at the exit orifice it will lose the necessary energy for atomization. This becomes particularly pronounced as viscosity approach surface tension, as a relatively more viscous formula would tend to slow down more at a bottle neck point then a less viscous formula. By valve stem orifice it is meant the orifice though which the concentrate passes. Some spray devices may have a second orifice in which only the compressed gas goes through.

TABLE 11
Ratio of Area stem/Area insert

| | | Area stem/Area | |
|---|---|---|---|
| Spray system | Example Table | actuator insert | Dv50 |
| COSTERECO ™ | Table 3 | 1 | 69.3 (Ex. 13) |
| Salvalco Eco-Valve ® | Table 4 | 8 | 82.5 (Ex. 23) |
| Lindal ® neo EMT | Table 5 | 21 | 377.9** (Ex. 33) |

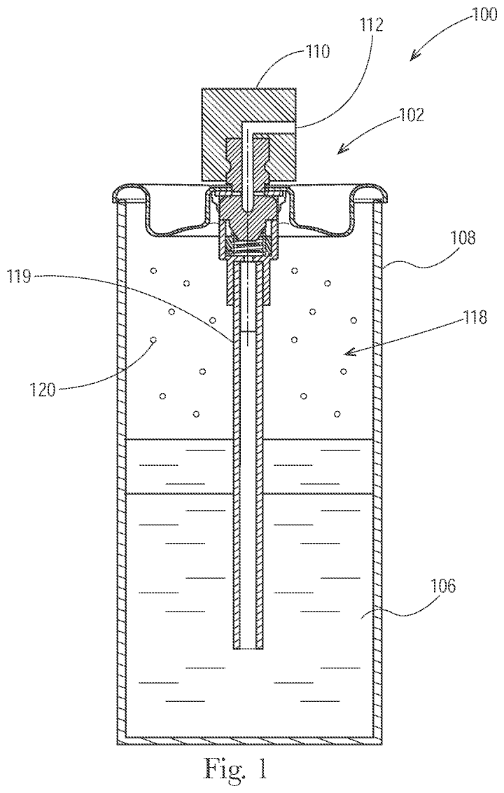

Referring to FIG. 1, one non-limiting example of a spray device that may help reduce clogging in some instances is shown. While it may be desirable to use the spray device shown in FIG. 1 to reduce the risk of clogging in some instances, it will be appreciated that other spray devices, including other types of actuators and valve assemblies, etc., may also be used. The spray device 100 comprises a container 102, a compressed gas propellant 120 and a composition 106. It will be appreciated that the propellant 120 and composition 106 are merely shown for purposes of illustration in FIG. 1, and FIG. 1 is not intended to limit in any way the type or arrangement of the propellant and composition within the container 102. The spray device 100 may be shaped and configured so that it is hand-holdable. The container 102 comprises a body 108, an actuator 110 having an actuator orifice 112, and a valve assembly 114 in fluid communication with a reservoir 118 storing the composition 106 and propellant 120. The reservoir 118 may be defined by one or more interior surfaces of the body 108. The reservoir may have a volume from about 20 ml, 40 ml, or 60 ml to about 220 ml, 210 ml, 200 ml, or 150 ml, the examples mean to be illustrative of the volume but not limiting. A dip tube 119 may extend into the reservoir 118 from the valve assembly 114. A gaseous propellant 120 will fill the headspace of the reservoir 118.

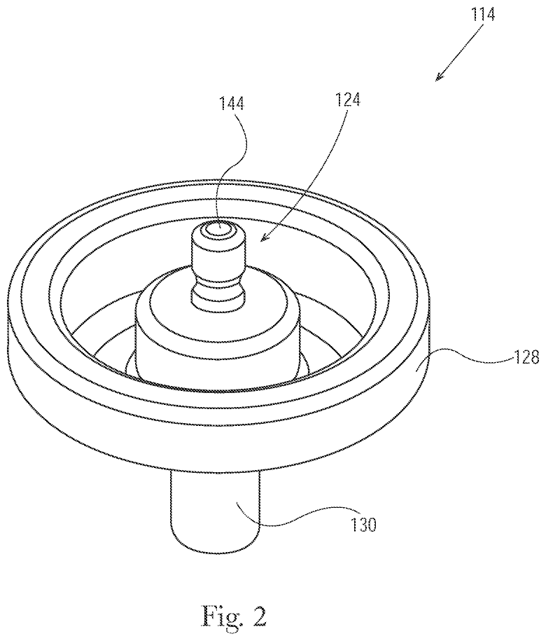

Referring to FIGS. 2 to 4, one non-limiting example of a valve assembly 114 which may be attached to the body 108 is shown. The valve assembly 114 comprises a slidably disposed valve stem 124 to which the actuator 110 attaches, a mounting flange 128 for attaching the valve assembly 114 to the body 108 (such as by crimping), and a housing 130 attached to the mounting flange 128. The valve assembly 114 also has an axial bore 144. The housing 130 may be attached by a variety of means to the mounting flange, as known in the art, including by a press fit, positive latching, welding, etc. The housing 130 contains a spring 132 that biases the valve stem 124. The spring 132 may comprise a plurality of coils. For effervescent bubbling valves please refer to U.S. Pat. No. 11,059,659.

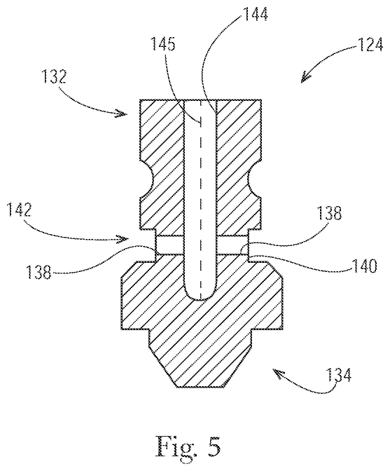

Turning to FIG. 5, the valve stem 124 comprises an upper portion 132 and a lower portion 134. The upper portion 132 has a distal end 136 and is configured to be attachable to the actuator 110. The lower portion 134 is configured to position at least a portion of the spring 132 there about. One or more valve stem orifices 138 (two being shown in the FIGS.) are disposed between the upper portion 132 and the lower portion 134. The valve stem orifices 138 are arranged in a radial direction with respect to the longitudinal axis 145 of the valve stem 124. The two or more valve stem orifices 138 open into a wall 140 of a groove 142 and communicate with an axial bore 144 that extends from the two or more valve stem orifices 138 to the distal end 136 of the upper portion 132. For effervescent valves the correspondent stem orifice is the one intended to expel the liquid composition, as shown by U.S. Pat. No. 11,059,659, specifically its example of stem orifice in FIG. 12a, reference number 124. It will be appreciated that the terms “radial” and “axial”, and derivatives thereof (e.g., radially and axially), are intended to merely refer to a general direction with respect to a feature or structure, and these terms are intended, unless expressly stated otherwise (e.g., solely axial or solely radial), to be fully inclusive of directions that are not purely radial or axial, such as substantially radial/axial directions and combinations of radial and axial directions where the net overall directional effect is more radial than axial or vice versa. The axial bore 144 in turn communicates with the actuator 110 when it is attached to the valve stem 124.

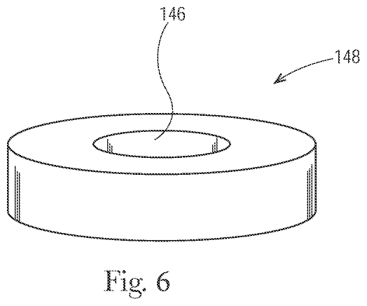

Referring to FIGS. 1, 4, and 6, mating sealing surfaces formed by an inner wall 146 of a substantially flat seal 148 and the wall 140 of the groove 142 form a valve that seals the valve stem orifices 138. The seal 148 may be formed from an elastomeric material, such as nitrile butadiene rubber (sometimes referred to as Buna-N). The seal 148 may be disposed about the valve stem and sandwiched between the mounting flange 128 and the housing 130, as shown by way of example in FIG. 2. The sealing surfaces are mated when the valve stem is not depressed, as shown in FIG. 2, thereby preventing flow of the composition and propellant mixture thru the valve stem orifices 138. When the actuator 110 is depressed, the sealing surfaces separate, thereby permitting the composition and propellant mixture to flow through the valve stem orifices 138 to the axial bore 144 and onto the actuator 110. As used herein, the term valve (as opposed to valve assembly) is intended to merely refer to the mating sealing surfaces that prevent flow of the composition and propellant mixture from the reservoir 118 to the actuator 110. The mating sealing surfaces may be provided in configurations other than shown in the FIGS. and described herein. In some specific embodiments, the valve may be a continuous flow valve, meaning there is flow through the valve for as long as the actuator is depressed. In contrast, a non-continuous or metered valve allows only predetermined amount of flow thru the valve regardless how long the actuator is depressed.

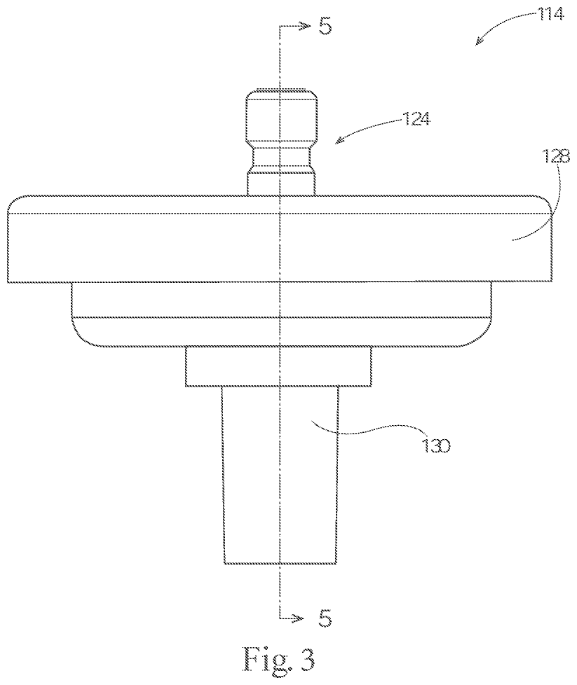

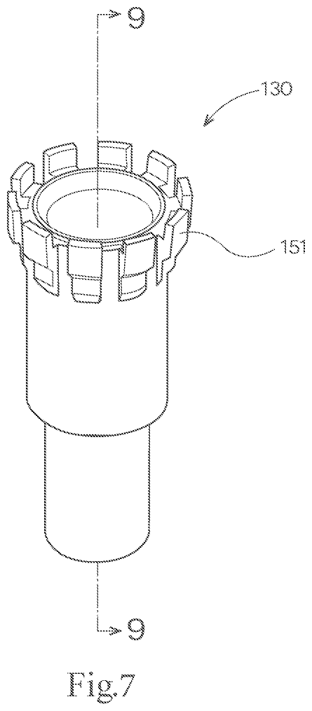

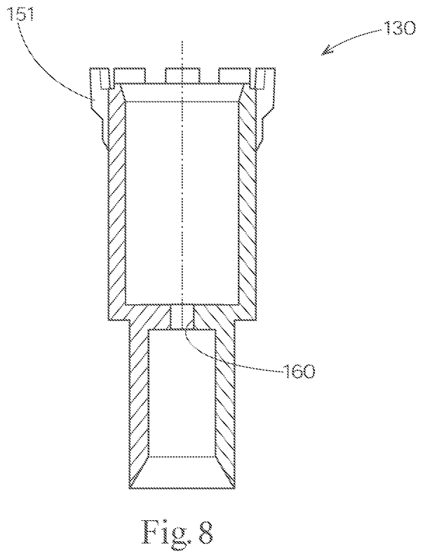

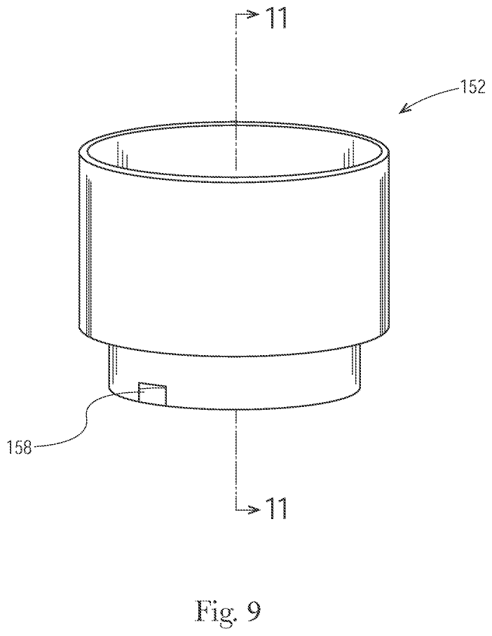

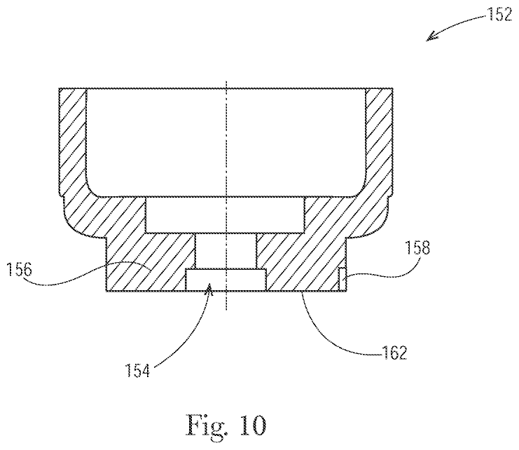

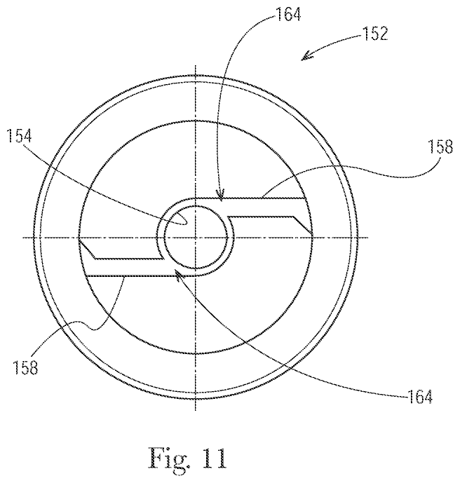

Referring to FIGS. 3, 4, and 7 to 11, the housing 130 has a plurality of fingers 151 for attaching the housing to the mounting flange 128. An insert 152, which in some embodiments may be cup-shaped, may be installed within the housing 130 between the dip tube and the valve stem 124. The insert 152 may be press-fit within the housing 130 or otherwise retained within the housing by other means known in the art. The insert 152 may receive one end of the spring 132. The insert 152 has an insert bore 154 disposed in a bottom wall 156 of the insert 152. The insert bore 154 is in fluid communication with the dip tube 119 and the interior of the insert 152 so that the composition and propellant mixture may flow from the dip tube 119 to the interior of the insert 152. The mixture then flows past the spring 132 and on to the valve.

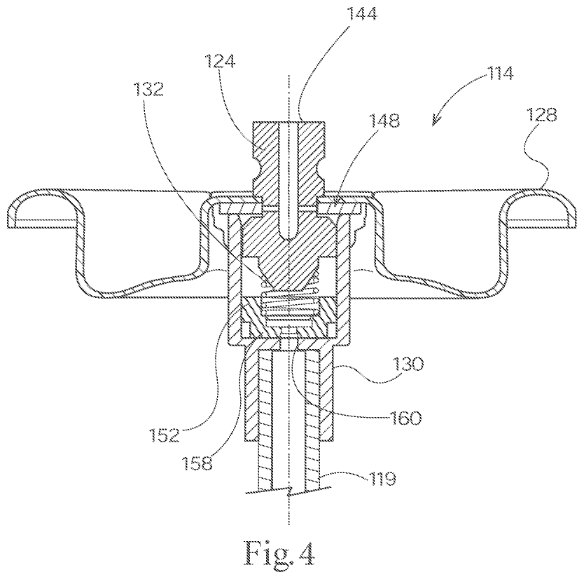

A plurality of passages 158 are disposed between the dip tube 119 and the distal end of the valve stem 124. While two passages are shown, one passage or more than two passages may be provided. The passages 158 are disposed adjacent the dip tube exit and/or the tail orifice 160 (FIG. 4), the tail orifice 160 being disposed just downstream of the dip tube exit. For purposes of clarity, the passages 158 of valve assembly 114 are considered to be disposed adjacent the dip tube 119 even though there is an intervening tail orifice 160 located between the dip tube exit and the passages 158. In an embodiment, the passages 158 may be tangentially disposed the insert bore 154. While the passages 158 are shown disposed in the bottom surface 162 of the insert 188, it is contemplated that the passages 158 may be provided by other structures/arrangements. In some specific embodiments, the passages may have a width of 0.01 inches and a height of 0.01 inches (0.25 mm) or a width of 0.01 inches (0.25 mm) and height of 0.013 inches (0.33 mm).

In compressed gas personal care products, to avoid over-dosing the product it is desirable that the spray device have a total mass flow rate of the composition plus compressed gas have a spray rate at most about 1 gram/sec and more preferably of about 0.3 grams/sec or from about 0.1 grams/sec to about 0.5 grams/sec, or from about 0.2 grams/sec to about 0.4 grams/sec, or from about 0.25 grams/sec to about 0.35 grams/sec.

### Emulsions

As mentioned, is well known from the art that many formulations' viscosity will far exceed the maximum possible surface tension of liquids, spray formulations with high viscosity are a desirable attribute to consumer, and that it is important that the viscosity of the final formulation exceed the surface tension in order to have acceptable aerosolization. Thus, it would be desirable to be able to spray such formulations from a compressed gas product. Surprisingly, the inventors have found that formulations are sprayable if they meet the following criteria:
1. They are formulations with two or more phases such as suspensions or emulsions
2. They are shear thinning
3. The surface tension of the continuous phase is higher than its viscosity

Emulsions and suspensions are typically shear thinning with the lowest viscosity possible being the viscosity of the continue phase; thus, the model of atomization in which the ratio of viscosity to surface tension is less than 1 still allows such formulations to spray.

Table 12 shows two formulations for emulsion concentrates, Examples 53 and 54. Table 13 shows the viscosities of the entire concentrates of Examples 53 and 54, and Table 14 shows the viscosity, surface tension, and ratio of viscosity to surface tension for the continuous phase of the emulsions of Examples 53 and 54.

Example 53 was prepared by placing in a suitable container (A) PPG-15, steareth-2, steareth-20 and heating the mixture to 70° C. while mixing until both steareths were melted. In a second container (B) the water was heated to 70° C. and stirred to form a robust vortex. When contents of container A were at 70° C. and all solids had melted, it was slowly poured into container B. Temperature was lowered to 60° C., and fragrance and water soluble emollients were added. Then aluminum chlorohydrate solution was added and the final product milled using IKA T50 for at least 3 min at 9000 rpm.

Example 54 was prepared by adding PEG-9 Polydimethylsiloxyethyl Dime (shin Etsu KF6028), isopropyl myristate, fragrance and cyclopentasiloxane to a suitable container and mixing to form a robust vortex, and call this phase, phase A. On a second container add the aluminum hydroxychloride 50% solution, water and dipropylene glycol, mix well until homogeneous and call this phase B. Add the first 25% of phase B into phase A dropwise while continuing to mix with a vigorous vortex. Add the remainder 75% of phase B to phase A as a weak stream. Once all phase B is added into phase A, mill the mixture for at least 3 min using IKA T50 at 9000 rpm.

Examples 53 may be a continuous phase for an oil-in-water emulsion in an inventive product with 10% emollient (propylene glycol with a viscosity of more than 20 cP). Example 54 may be a continuous phase for a water-in-oil emulsion in an inventive product with about 42% emollient.

TABLE 12
Emulsion Concentrates Ex. 53-54

| | Ex. 53 | Ex. 54 |
|---|---|---|
| Ingredients | (wt. %) | (wt. %) |
| Aluminum Hydroxychloride 50% Solution | 26.00 | 40.00 |
| Water | 57.00 | 10.00 |
| PPG-15 Stearyl Ether | 3.00 | |
| Steareth-2 | 1.90 | |
| Steareth-20 | 1.10 | |
| Propylene glycol | 10.00 | |
| Dipropylene Glycol | | 5.00 |
| PEG-9 Polydimethylsiloxyethyl Dime | | 2.00 |
| Isopropyl myristate | | 5.00 |
| Cyclopentasiloxane | | 37.00 |
| Fragrance | 1.00 | 1.00 |

TABLE 13
Finished Product Viscosity Ex. 53-54

| | Ex. 53 | Ex. 54 |
|---|---|---|
| Concentrate Formation Viscosity (cP) | 1200.00 | 43.75 |

Table 14 shows the continuous phase viscosity and surface tension for examples 53-54. For example 53, the continuous phase includes water, antiperspirant active solution, and dipropylene glycol; for example 54, the continuous phase includes isopropyl myristate, cyclopentasiloxane and fragrance.

TABLE 14

| Continuous Phase | Ex. 53 | Ex. 54 |
|---|---|---|
| Viscosity (cP) | 7.25 | 6.25 |
| Surface tension (dyn/cm) | 59.80 | 19.30 |
| Viscosity/surface tension | 0.12 | 0.32 |

Examples 53 and 54 were each placed into a 45 mm×150 mm aluminum aerosol can, which was then fitted with a valve, crimped, and pressurized with nitrogen to 100±5 psi. Samples were prepared in such a way that, for example, example 55 used 100 g of example 53 formulation and example 56 used 100 g of example 54 formulation. Examples 55 and 56 used Coster® spray system COSTERECO™ using a KV valve with no vapor tap and one stem orifice of 0.010 inch and a tail orifice of 0.042 inch. A Coster® actuator with a 0.010 inch mechanical breakdown insert and a stem volume reducer center post that fits into the valve stem ID. The results in Table 15 indicate that with the ratio of viscosity to surface tension below 1, good atomization and spraying was achieved.

TABLE 15
Particle size analysis results for emulsions sprayed with
compressed nitrogen and Coster Eco-Valve ® and actuator

| | Ex. 55 | Ex. 56 |
|---|---|---|
| Dv50 μm | 68.15 | 100.53 |

### Emollients

The concentrate composition can comprise an emollient system including at least one emollient, but it could also be a combination of emollients. Suitable emollients are often liquid under ambient conditions. Depending on the type of product form desired, the amount of emollient(s) in the concentrate composition may be at least about 10%, by weight of the concentrate. In some embodiments, the concentrate may comprise one or more emollients from about 10% to about 80%, by weight of the concentrate, and in other embodiments, the concentrate may comprise one or more emollients, by weight of the concentrate, at least about 20%, at least about 25%, or from about 20% to about 80%, or from about 25% to about 80%, or even be 100% of the concentrate.

For some products, the composition may comprise an emulsion, either an oil-in-water emulsion or a water-in-oil emulsion. If there is an emulsion, the continuous phase may comprise a concentrate comprising one or more emollients.

In some cases, the product comprises a composition comprising a water-in-oil emulsion comprising a continuous phase, wherein the continuous phase comprises a concentrate comprising at least about 10%, by weight of the mixture, of one or more water-insoluble emollients. In some embodiments, the concentrate in the continuous phase may comprise one or more water-insoluble emollients from about 10% to about 80%, by weight of the concentrate, and in other embodiments, the concentrate may comprise one or more water-insoluble emollients, by weight of the concentrate, at least about 20%, at least about 25%, or from about 20% to about 80%, or from about 25% to about 80%, or even be 100% of the concentrate.

In some cases, the product comprises a composition comprising an oil-in-water emulsion comprising a continuous phase, wherein the continuous phase comprises a concentrate comprising at least about 10%, by weight of the mixture, of one or more water-soluble emollients. In some cases, the continuous phase may comprise from about 10% to about 40%, by weight of the concentrate, of at least one water-soluble emollient, in other cases from about 15% to about 40%, from about 20% to about 40%, or from about 20% to about 40%, or from 10% to 50%.

In some cases, such as when the concentrate is a single phase, all the emollients or at least one of the emollients may have a viscosity of at least about 20 cP. The higher viscosity emollients, such as those with viscosity of at least 20 cP, are thicker emollients and provide good consumer skin feel. For products with a single phase concentrate, the one or more emollients may be water-soluble or water-insoluble or some combination thereof. In cases in which an emulsion comprises a water-insoluble emollient, at least one water-insoluble may have a viscosity of at least about 20 cP. In cases in which an emulsion comprises at least one water-soluble emollient, at least one emollient may have a viscosity of at least about 10 cP, in some cases at least about 5 cP.

Emollients suitable for use in the concentrate compositions can be either water soluble or water insoluble. Water soluble emollients are identified as able to form a single phase mixture with water, with light transmission of the sample of greater than about 95%, as measured in the test method described herein. Water soluble emollients include, but are not limited to, propylene glycol, polypropylene glycol (like dipropylene glycol, tripropylene glycol, etc.), diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols. Water-insoluble emollients include, but are not limited to, volatile silicone emollients such as cyclopentasiloxane, nonvolatile silicone emollients such as dimethicone, mineral oils, petrolatum, water insoluble alkyl ethers, esters, carbonates, low melting point triglycerides and combinations thereof. One example of a suitable water insoluble emollient comprises PPG-15 stearyl ether, isopropyl myristate, caprylic capric triglyceride and dipropyl heptyl carbonate. Other examples of suitable water soluble emollients include dipropylene glycol, glycerol, and propylene glycol.

Solubility of an emollient in water may be determined by measuring the amount of light transmittance (a light transmittance value) through a simple mixture of water and emollient at the same weight/weight concentrations as in a final concentrate composition. For example, the solubility of an emollient at a concentration of 19% w/w in a final concentrate composition comprising water having a concentration of 38% w/w can be determined by measuring the light transmittance of an emollient at 19% w/w concentration in just water. Light transmittance may be measured using a spectrophotometer, such as, for example, a Genesys 10 Vis Spectrophotometer available from Thermo Electron Corp (USA), wherein a light transmittance value greater than 95% at 25° C. indicates sufficient solubility in water.

### Deodorant Actives

Suitable deodorant actives can include any topical material that is known or otherwise effective in preventing or eliminating malodor associated with perspiration. Suitable deodorant actives may be selected from the group consisting of antimicrobial agents (e.g., bactericides, fungicides), malodor-absorbing material, and combinations thereof. For example, antimicrobial agents may comprise cetyl-trimethylammonium bromide, cetyl pyridinium chloride, benzethonium chloride, Di isobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

### Odor Entrappers

The composition can include an odor entrapper. Suitable odor entrappers for use herein include, for example, solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms; therefore, this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

Oil in Water Emulsion and Water in Oil Emulsion

The concentrate compositions of the present invention may comprise an emulsion. An emulsion is a two or multiple phase composition in which there is a continuous phase and a dispersed phase. An oil in water emulsion consists of an oil phase that is dispersed into a continuous water phase. The oil phase may comprise of an emollient, an emulsifier, and optionally other ingredients such as, but not limited to, co-emulsifiers, fragrances, deodorant actives, skin conditioners, or other oil soluble ingredients. Emollients in the oil phase are water insoluble liquids that smooth, soften, or lubricate the skin and will typically comprise more than 30% of an oil phase. The role of the oil phase in the composition is multifold. An oil phase must be water insoluble enough to provide a stable emulsion, not interfere with the antiperspirant active, provide a solvent system for the fragrance, and provide a lubricious soft feel to the consumer's skin throughout the day.

The continuous phase of the oil in water emulsion may be comprised of water soluble emollients, deodorant actives, and antiperspirant actives. The actives and emollients needs to be water soluble enough to form a stable emulsion.

A water in oil emulsion consists of a water phase that is dispersed into a continuous oil and or silicone phase. The water phase may comprise of one or more water soluble emollients, an emulsifier, and optionally other ingredients such as, but not limited to, co-emulsifiers, deodorant actives, skin conditioners, antiperspirant active and or other water soluble ingredients. The actives and emollients needs to be water soluble enough to form a stable emulsion.

The continuous phase of the water in oil emulsion is comprised of water insoluble emollients, liquids that smooth, soften, or lubricate the skin and will typically comprise more than 10-50% the concentrate. As per oil in water emulsion, the role of the oil phase in the water in oil composition is multifold. An oil phase must be water insoluble enough to provide a stable emulsion, not interfere with the antiperspirant active, provide a solvent system for the fragrance, and provide a lubricious soft feel to the consumer's skin throughout the day.

### Emulsifiers, Surfactants and Co-Emulsifiers

The multi-phase concentrate such as an emulsion may also comprise one or more of the following: emulsifiers, and co-emulsifiers. These materials may perform several functions in the emulsion concentrate composition including, but not limited to, stabilizing the emulsion, solubilization of the actives and fragrance. Choice of these materials is dependent on the composition of the emulsion, particularly whether the composition is a water in oil or an oil in water emulsion. Moreover, it is desirable to choose materials that would not adversely impact surface tension by lowering it below the viscosity of the continuous phase. Furthermore, the choice of any emulsifier, surfactant, or co-emulsifier must not interfere with the performance of the antiperspirant or deodorant actives used in the antiperspirant or deodorant composition. For example, the use of some anionic surfactants can interfere with efficacy of cationic aluminum antiperspirant actives via the formation of insoluble ion pairs. Lastly, it is appreciated that different oil phase emollients will require different emulsifiers, surfactants, or co-emulsifiers to create a stable multiphase emulsion. Said differently, the different oil phases that are insoluble in one another may require different emulsifiers and co-emulsifiers to stabilize each emulsion phase in the emulsion.

An emulsifier or co-emulsifier used to stabilize an emulsion may be chosen based on the required HLB (hydrophilic lipophilic balance) of the oil phase emollients. The HLB of a surfactant is a measure of the ratio of the hydrophobic to the hydrophilic portion of the surfactant or emulsifier. Choice of the desired HLB for an emollient is dependent on the emollient or emollient blend polarity and structure. The use of the HLB system for emulsion formulation is discussed in the following references:
1. Griffin WC; Calculation of HLB Values of Non-Ionic Surfactants, Journal of the Society of Cosmetic Chemists; 1954. Vol. 5, pp 249-235
2. Vaughan, C. D. Rice, Dennis A.; Predicting O/W Emulsion Stability by the “Required HLB Equation”; Journal of Dispersion Science and Technology; 1990. Vol. 11 (1), pp 83-91.

Any known emulsifier, or co-emulsifier can be used in an aerosol concentrate antiperspirant or deodorant compositions herein, provided that they stabilize the emulsions and do not interfere with the delivery or action of the antiperspirant or deodorant actives. Suitable classes of emulsifiers and surfactants include anionic, cationic, and nonionic materials. Moreover, for some oil phase emollients, polymeric emulsifiers and surfactants are suitable. In some embodiments, nonionic emulsifiers, and co-emulsifiers are preferred to prevent interaction with any charged antiperspirant active (i.e., aluminum chlorohydrate) or deodorant active (i.e., benzethonium chloride).

Emulsifier, and co-emulsifier concentrations will vary based on composition and level of the discontinuous, however it is generally found to be desirable to not have excess emulsifier, and co-emulsifier to prevent lowering the surface tension of the continuous phase and increase particle size of spray. Total levels of emulsifier, surfactant, and co-emulsifier should be less than about 12%, more preferably less than about 7% and most preferably less than about 3%, by weight of the antiperspirant or deodorant composition for an oil in water emulsion. Total levels of emulsifier, surfactant, and co-emulsifier should be less than about 5%, more preferably less than about 3% and most preferably less than about 1%, by weight of the antiperspirant or deodorant composition for water in oil emulsion.

Suitable nonionic emulsifiers and co-emulsifier include, but are not limited to, linear saturated and unsaturated C12 to C30 primary alcohols that are etherified with 1 to 100 ethylene oxide units per molecule. More preferred nonionic emulsifiers laureth, trideceth, myristeth, ceteth, ceteareth steareth, arachideth, and beheneth, having respectively 1 to 100 ethylene oxide units per molecule. Some examples of preferred nonionic emulsifiers include, but are not limited to, steareth-1, steareth-2, steareth-3, steareth-20, steareth-21, steareth-100, ceteareth-10. ceteareth-20 ceteareth-30, ceteth-1, ceteth-2, ceteth-3, ceteth-10, myristeth-1, myristeth-2, laureth-4, beheneth-2, beheneth-3, and beheneth-5, behenth-10, and beheneth-25.

In some embodiments of the present invention, steareth-2 and steareth-21 are preferred emulsifiers or surfactants. Preferred weight ratios of steareth-21 to steareth-2 range from about 0.2 to about 5, more preferably from about 0.2 to about 2. In some embodiments of the present invention Ceteth-10 and Laureth-4 are preferred emulsifiers or co-emulsifiers.

Suitable nonionic polymer emulsifiers and co-emulsifiers include but are not limited to particularly desirable class of emulsifiers for water in oil emulsions comprises dimethicone copolymers, namely polyoxyalkylene modified dimethylpolysiloxanes. The polyoxyalkylene group is often a polyoxyethylene (POE) or polyoxypropylene (POP) or a copolymer of POE and POP. The copolymers also include Cl to C12 alkyl groups as functional groups. Examples of suitable surfactants include DC5225 and DC 5200 (from Dow Corning), Abil EM 90 and EM 97 (from Gold Schmidt) and KF 6026, KF 6028, KF 6038 (from Shinetsu Silicones).

Suitable cationic emulsifiers and surfactants include, but are not limited to, distearyldimonium chloride, behentrimonium chloride and palmitamido-propyltrimonium chloride.

Suitable co-emulsifiers include, but are not limited to, fatty alcohols such as stearyl alcohol, cetyl alcohol, and cetearyl alcohol. However, in some embodiments, the emulsion may not comprise a fatty alcohol, as fatty alcohols can increase the viscosity of the oil in water emulsion to a level that is difficult to spray, making them undesirable.

Chelator

The compositions may comprise a chelator. Specific and/or additional chelators in the present invention may include, but are not limited to, diethylenetriaminepentaacetic acid (DTPA), diethylenetriaminepentakis (methylenephosphonic acid) (DTPMP), desferrioxamine, their salts and combinations thereof, EDTA, DPTA, EDDS, enterobactin, desferrioxamine, HBED, and combinations thereof. The amount of chelant, by weight of composition, may be from about 0.05% to about 4%.

Preservatives

The composition can include a preservative. The preservative is included in an amount sufficient to prevent spoilage or prevent growth of inadvertently added microorganisms for a specific period of time, but not sufficient to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115@ from Sutton Laboratories, Inc.; polymethoxy bicyclic oxazolidine, available under the trade name Nuosept® C from Huls America; formaldehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative can range from about 0.0001% to about 0.5%, alternatively from about 0.0002% to about 0.2%, alternatively from about 0.0003% to about 0.1%, by weight of the composition.

### Solvents

In this embodiment solvents are liquids intended to dissolve, break down, and/or disperse other ingredients in the formulation, examples of such include, but are not limited to, ethanol, water, isopropyl alcohol, polydecene, decane, isodecane, 1-decene, 1-heptanol, 1-hexanol, 1-hexene, 1-methoxy-2-propanaol acetate, 1-octene, 2,2,4-trimethylpentane, 2-butanone, 2-butoxyethanol, 2-ethoxyethnol. For single phase products, the amount of solvent may range from about 0% to about 90%, by weight of the concentrate, or from about 20% to about 80%, from about 30%, about 40%, about 50% to about 55%, about 65%, or about 75%, by weight of the concentrate. For products with water-in-oil emulsions or oil-in-water emulsions, the continuous phase may comprise from about 0% to about 90% solvent, by weight of the concentrate, or from about 20% to about 80%, from about 30%, about 40%, about 50% to about 55%, about 65%, or about 75%, by weight of the concentrate.

### Fragrance

One or more fragrance materials are included to help cover or mask malodors resulting from perspiration, or which otherwise provide the compositions with the desired perfume aroma. These fragrance materials may include any perfume or perfume chemical suitable for topical application to the skin.

The concentration of the fragrance in the spray concentrate antiperspirant or deodorant compositions should be effective to provide the desired aroma characteristics or to mask malodor wherein the malodor is inherently associated with the composition itself or is associated with malodor development from human perspiration. Compositions of the present invention may comprise fragrances selected from the group consisting of free perfumes, encapsulated perfumes, and mixtures thereof. The total perfume may include one or more individual perfume chemicals provided that the perfume can emit a detectable perfume odor or can mask or help to mask odors associated with perspiration. Generally, the deodorant compositions of the present invention may comprise the total perfume at concentrations ranging from about 0.05% to about 10%, preferably from about 0.5 to about 5% and more preferably from about 1 to about 4%. As previously discussed, the choice of fragrance level and the emollient level both in the first oil phase are often related by desired emollient to fragrance weight ratios of from about 1:1 to about 10:1, and more preferably, from 3:1 to about 7:1. The fragrance that is in the antiperspirant and/or deodorant composition may be entirely in the first oil phase and may be solubilized in the first oil phase.

Nonlimiting examples of fragrance materials suitable for use as a free perfume or an encapsulated perfume include any known fragrances in the art or any otherwise effective fragrance materials. Typical fragrances are described in Arctander, Perfume and Flavour Chemicals (Aroma Chemicals), Vol. I and II (1969) and Arctander, Perfume and Flavour Materials of Natural Origin (1960). U.S. Pat. No. 4,322,308, issued to Hooper et al., Mar. 30, 1982 and U.S. Pat. No. 4,304,679, issued to Hooper et al., Dec. 8, 1981 disclose suitable fragrance materials including, but not limited to, volatile phenolic substances (such as iso-amyl salicylate, benzyl salicylate, and thyme oil red), essence oils (such as geranium oil, patchouli oil, and petitgrain oil), citrus oils, extracts and resins (such as benzoin siam resinoid and opoponax resinoid), “synthetic” oils (such as Bergamot™ 37 and Bergamot™ 430, Geranium™ 76 and Pomeransol™ 314); aldehydes and ketones (such as B-methyl naphthyl ketone, p-t-butyl-A-methyl hydrocinnamic aldehyde and p-t-amyl cyclohexanone), polycyclic compounds (such as coumarin and beta-naphthyl methyl ether), esters (such as diethyl phthalate, phenylethyl phenylacetate, non-anolide 1:4).

Suitable fragrance materials may also include esters and essential oils derived from floral materials and fruits, citrus oils, absolutes, aldehydes, resinoides, musk and other animal notes (e.g., natural isolates of civet, castoreum and musk), balsamic, and alcohols (such as dimyrcetol, phenylethyl alcohol and tetrahydromuguol). For example, the present invention may comprise fragrances selected from the group consisting of decyl aldehyde, undecyl aldehyde, undecylenic aldehyde, lauric aldehyde, amyl cinnamic aldehyde, ethyl methyl phenyl glycidate, methyl nonyl acetaldehyde, myristic aldehyde, nonalactone, nonyl aldehyde, octyl aldehyde, undecalactone, hexyl cinnamic aldehyde, benzaldehyde, vanillin, heliotropine, camphor, para-hydroxy phenolbutanone, 6-acetyl 1,1,3,4,4,6 hexamethyl tetrahydronaphthalene, alpha-methyl ionone, gamma-methyl ionone, amyl-cyclohexanone, and mixtures thereof.

### Antiperspirant and Deodorant Actives

The compositions and products of the present invention may be antiperspirant and/or deodorant products and compositions. The water phase of the emulsions generally includes water and an antiperspirant active and/or a deodorant active dissolved in water. The concentration of the antiperspirant active and/or deodorant actives in the composition should be sufficient to provide the finished antiperspirant or deodorant composition with the desired perspiration wetness and/or odor control benefits.

Exemplary antiperspirant active concentrations range include from about 0.1% to about 26%, from about 1% to about 20%, and from about 2% to about 10%, by weight of the composition. All such weight percentages are calculated on an anhydrous metal salt basis exclusive of water and any complexing or buffering agent such as, for example, glycine, glycine salts or other amino acids and any stabilizing agents such as calcium chloride, calcium salts, or strontium salts.

Preferred aluminum salts are those having the general formula Al₂(OH)₆₋ₐXₐ wherein X is Cl, Br, I or NO₃, and a is about 0.3 to about 5, preferably about 0.8 to about 2.5. Preferred actives in this group include, but are not limited to, aluminum chlorohydrate (ACH) wherein a is from about 1 and the mole ratio of Al/Cl is from about 1.9 to about 2.1. Aluminum sesquichlorohydrate (ASCH) wherein a is from about 1.05 to about 1.61 and the mole ratio of Al/Cl is from about 1.26 to about 1.89, and aluminum dichlorohydrate (AD-CH) wherein a is from about 1.6 to about 2.2 and the mole ratio of Al/Cl is from about 0.9 to about 1.25.

Enhanced efficacy aluminum chlorohydrate is referred to as “ACH” herein.

The ACH salts used in the present invention may also include soluble calcium salts. Soluble calcium salts are those calcium salts that are soluble in water or that dissolve in the aqueous solution of antiperspirant salt (i.e., a solution of the aluminum salt and/or zirconium salt). Calcium salts which may be utilized are any of those which do not otherwise interfere with the solubility or effectiveness of the antiperspirant salt. Preferred calcium salts include calcium chloride, calcium bromide, calcium nitrate, calcium citrate, calcium formate, calcium acetate, calcium gluconate, calcium ascorbate, calcium lactate, calcium glycinate and mixtures thereof. Calcium carbonate, calcium sulfate and calcium hydroxide may also be used because they will dissolve in an aqueous solution of the antiperspirant salt.

The ACH salts used in the present invention may also contain a water soluble amino and/or hydroxy acid which is effective in increasing and/or stabilizing the HPLC peak 4:3 area ratio of the antiperspirant salt. Such acids include amino- and/or hydroxy-substituted lower alkanoic acids (including substituted derivatives thereof), preferably where the amino or hydroxy group is located on the a-carbon (i.e., the same carbon to which the carboxy group is attached). The lower alkanoic acid will generally have 2 to 6, preferably 2 to 4, carbon atoms in the alkanoic acid chain. Typical amino and/or hydroxy substituted lower alkanoic acids include any of the amino acids such as glycine, alanine, valine, leucine, isoleucine. P-alanine, serine, cysteine, β-amino-n-butyric acid, γ-amino-n-butyric acid, etc. and hydroxy acids such as glycolic acid and lactic acid. These amino and/or hydroxy substituted lower alkanoic acids may also contain various substituents which do not adversely affect their activity. The preferred amino and/or hydroxy substituted lower alkanoic acids are glycine, alanine, and glycolic acid, with glycine being most preferred.

In some embodiments, a preferred active is an aqueous solution of ADCH that also contains calcium chloride and glycine. The preferred ADCH with calcium chloride and glycine is further characterized by having more than 50% peak 4 and 5 as measured by HPLC, a Al:Cl molar ratio of about 0.9 to about 1.25, an Al to glycine wt ratio of about 1.7 to 7.7, and calcium to glycine wt ratio of about 0.1 to about 1.5. In some embodiments a preferred active is an aqueous solution of ASCH that also contains calcium chloride and glycine. The preferred ASCH with calcium chloride and glycine is further characterized by having more than 35% peak 4 and 5 as measured by HPLC, a Al:Cl molar ratio of about 1.26 to about 1.89, an Al to glycine wt ratio of about 4 to 10, and calcium to glycine ratio of about 0.1 to about 1.5.

The antiperspirant or deodorant compositions provided herein may comprise a non-aluminum antiperspirant active. Suitable non-aluminum antiperspirant actives include, but are not limited to, oxybutynin chloride, chitotosan, PVM/MA polymers, calcium channel blockers, gingerol, liquid fatty acid and metal ion combinations, magnesium gluconate, silicic acid, silicic acid salts, and vicinal diols such as propylene glycol.

The antiperspirant and/or deodorant compositions provided herein may comprise a deodorant active, alternatively meaning that a deodorant active is substituted for an antiperspirant active or used in addition to the antiperspirant active. Some deodorants may not have an antiperspirant active and/or may be substantially free or free of aluminum. Suitable deodorant actives may be selected from the group consisting of antimicrobial agents (e.g., bactericides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof.

In some embodiments, antibacterials (deodorant actives) may be selected from the group consisting of 2-Pyridinol-N-oxide (piroctone olamine), lupamin, beryllium carbonate, magnesium carbonate, calcium carbonate, magnesium hydroxide, magnesium hydroxide and magnesium carbonate hydroxide, partially carbonated magnesium hydroxide, potassium carbonate, potassium bicarbonate, sodium carbonate, sodium sesquicarbonate, baking soda, hexamidine, zinc carbonate, thymol, polyvinyl formate, salycilic acid, niacinamide and combinations thereof.

The concentration of the optional other active(s) may range, individually or cumulatively, from about 0.001%, from about 0.01%, of from about 0.1%, by weight of the composition to about 20%, to about 10%, to about 5%, or to about 1%, by weight of the composition.

### Compressed Gases

Suitable compressed gas propellants include, but are not limited to, nitrogen, air, carbon dioxide, nitrous oxide, argon, helium, and oxygen, and combinations thereof, they are in the gas phase at 25° C. and at 725 psi. Suitable compressed gas propellants are further discussed in U.S. Pat. No. 11,059,659, the substance of which is herein incorporated by reference.

Pressure:

The amount of compressed gas in a product should be high enough to be able to dispense the entire contents of the product in the container. The amount of gas in the container is dependent on pressure and the volume of container that is not occupied by the formulation. Without intending to be bound by any theory, if the volume occupied by a formula is too small, consumers will have the impression that the product canister may be mostly empty. Thus, the volume of the formula should not be below about 50% of the total volume of the container and preferably at least about 60% of total volume of the container and more preferably be about or at least about 70% of the container. Pressure of the product should not exceed safety bursting level of the container. For example, United States of America Department of transportation (DOT) general pressure conditions mandate that the pressure inside the container may not exceed 180 psig at 54.4° C. (130° F.) and the metal container must be capable of withstanding without bursting a pressure of at least one and one-half times the equilibrium pressure of the contents at 54.4° C. (130° F.).

TABLE 16
Authorized Metal Aerosol Containers

| If the gauge pressure (psig) | |
|---|---|
| at 54.4° C. (130° F.) is . . . | Authorized container |
| 140 or less | Non-DOT specification, DOT 2P, |
| | DOT 2Q, DOT 2Q1. |
| Greater than 140 but not exceeding 160 | DOT 2P, DOT 2Q, DOT 2Q1. |
| Greater than 160 but not exceeding 180 | DOT 2Q, DOT 2Q1. |
| Not to exceed 210 | DOT 2Q1 (Non-flammable only). |

Without intending to be bound by any theory, the product pressure should be high enough to provide a particle size that is preferred by the consumer; for example, if pressure is too high, the particle size of a product may be too small and the consumer may perceive that the product has too much of a bloom and or is cloudy. Thus, the preferred pressure should be high enough to expel all the contents of the container and should not exceed safety and regulatory conditions. Pressure should be not lower than about 70 psi and preferably no lower than about 100 psi and should not exceed about 140 psi.

### Gas Solubility

As discussed herein, unlike liquefied propellants the % w/w amount of compressed gas in the final composition depends on how much empty space is left in the can after the can is filled with the formula concentrate. As product is dispensed, the amount of space in the spray container that is not occupied by formula increases and that leads to a drop of pressure across the life of the product. In consequence, the amount of product dispensed and particle size of aerosol varies thru the life of the product, with amount dispensed per second decreasing and particle size increasing. Without intending to be bound by any theory, personal care products are better received by consumers if throughout the life of the product the consumers can experience a relatively constant benefit. It was found that in a spray product using compressed gases, if the gas or mixture of gases used as propellant presents sufficient large amount of solubility in the formula, as the product is dispensed, it has the ability to replenish some of the lost pressure, thus reducing the decay of the spray performance through the life of the product.

In Tables A and B, below, are the Examples A, B, and C and their concentrates. Gas solubility for each concentrate was measured according to the Gas Solubility Test Method, described herein. Each of the concentrates, 103 g, were placed into a 45 mm×162 mm aluminum aerosol can, which was then fitted with a Coster® KV valve with no vapor tap and crimped and vacuum to −14 mmHg. Gas was introduced to the aerosol canister thru the valve using a 500 mL burette. The burette was pressurized to 130 psi with the respective gases as per example A-C tables. The can were fitted to the burette, which was then opened to allow gas to enter the containers. The containers were allowed to equilibrate for 10-15 seconds then they were removed and shaken for 10 seconds. This action was repeated three times for each container resulting on the initial pressures in Table C. Product were characterized for their initial pressure, dispensing rate, and particle size. Product were placed on a water bath at 21° C. for at least 5 min before sprayed. Each sample were sprayed for 4 seconds, returned to the water bath for equilibration. This process was done until each container had dispensed 75% of its fill weight (gas+formula).

As can be observed in Tables C and D, below, the increase in solubility from example A to B had a directionally better impact on the decay of the spay quality; however, the nearly 5 fold increase from sample B to C demonstrates that it is possible, even when starting at lower pressure as example C did, to better maintain the spray quality thru the life of the product.

TABLE A
Examples A, B, and C

| | Ex. A | Ex. B | Ex. C |
|---|---|---|---|
| | (W/W %) | (W/W %) | (W/W %) |
| Ethanol 200 proof | 53.70 | 64.69 | 61.18 |
| Dipropylene Glycol | 37.53 | 18.77 | 17.75 |
| Triethyl Citrate | 0.99 | | |
| Caprylic/Capric Triglyceride | | 9.88 | 9.34 |
| (Miritol ® 318 from BASF ®) | | | |
| Citric Acid | 0.99 | | |
| Purified Water | 3.59 | 3.46 | 3.27 |
| Fragrance | 1.98 | 1.98 | 1.87 |
| Nitrogen | 1.23 | 1.23 | |
| Carbon Dioxide | | | 6.60% |

TABLE B
Examples A, B, and C Concentrate

| | Ex. A | Ex. B | Ex. C |
|---|---|---|---|
| | Concentrate | Concentrate | Concentrate |
| Ingredient | (W/W %) | (W/W %) | (W/W %) |
| Ethanol 200 proof | 54.37 | 65.50 | 65.50 |
| Dipropylene Glycol | 38.00 | 19.00 | 19.00 |
| Triethyl Citrate | 1.00 | | |
| Caprylic/Capric Triglyceride | | 10.00 | 10.00 |
| (Miritol ® 318 from BASF ®) | | | |
| Citric Acid | 1.00 | | |
| Purified Water | 3.63 | 3.50 | 3.50 |
| Fragrance | 2.00 | 2.00 | 2.00 |

TABLE C

| | Gas | | Pressure @ | Delta | | Dispensing | Delta |
|---|---|---|---|---|---|---|---|
| | Solubility in | | 75% of | Pressure vs | | rate @ 75% | dispensing |
| | Concentrate | Initial | product | 75% | Initial | of product | rate vs 75% |
| | H | Pressure | dispensed | dispensed | Dispensing | dispensed | dispensed |
| | (mol/m³Pa) | (PSI) | (PSI) | (PSI) | rate (g/s) | (g/s) | (g/s) |
| Ex. A | 2.70E−05 | 113 | 57 | 56 | 0.43 | 0.25 | 0.18 |
| Ex. B | 8.42E−05 | 112 | 61 | 51 | 0.45 | 0.29 | 0.16 |
| Ex. C | 4.11E−04 | 100 | 67 | 33 | 0.32 | 0.28 | 0.04 |

TABLE D

| | Gas Solubility in | Initial | | Delta |
|---|---|---|---|---|
| | Concentrate H | DV<50> | DV<50> @ 75% | DV<50> vs 75% |
| | (mol/m³Pa) | (μm) | dispensed (μm) | dispensed (μm) |
| Ex. A | 2.70E−05 | 60.00 | 82.33 | −22.33 |
| Ex. B | 8.42E−05 | 62.93 | 81.58 | −18.65 |
| Ex. C | 4.11E−04 | 59.68 | 65.01 | −5.33 |

### Method of Making

Single phase compositions are generally prepared by mixing of the ingredients in a standard manner until complete dissolution of the ingredients was achieved. If necessary, a milling step is added at the end to homogenize the mixture.

The oil-in-water emulsion are in general prepared by mixing the oil phase, water insoluble emollients, water insoluble actives and the emulsifier in one container and if necessary, heating to solubilize and or melt the emulsifier. The water phase and the water soluble actives and emulsifiers were placed in a second containers and if necessary heated to match the temperature of the oil phase. Once both phases are ready, they were mixed under standard conditions and if necessary milled in a high shear homogenizer to achieve a small emulsion particle size distribution.

The water-in-oil emulsions and are made in the following manner. The materials in the aqueous phase such as antiperspirant actives, water soluble deodorant actives and water soluble emollients are mixed using conventional mixing techniques. If necessary for some of the deodorant actives, after all ingredients have been added, the pH of the aqueous phase is adjusted with HCl or NaOH to a pH between 3.5 and 4.0. The silicone phase is mixed using conventional mixing techniques. To create the emulsion, the water phase is added in a dropwise fashion via a separation funnel to the oil phase with strong agitation of the silicone phase using an overhead mixer with mixing blade. After all the water phase has been added to the formulation, the emulsion is subsequently milled in a high shear homogenizer to achieve a small emulsion particle size distribution

### Test Methods

The term “viscosity” means dynamic viscosity (measured in centipoise, cPs, or Pascal-second, Pa-s) or kinematic viscosity (measured in centistokes, cst, or m2/s) of a liquid at approximately 25° C. and ambient conditions. Dynamic viscosity may be measured using a rotational viscometer, such as a Brookfield Dial Reading Viscometer Model 1-2 RVT available from Brookfield Engineering Laboratories (USA) or another substitutable model known in the art. Typical Brookfield spindles which may be used include, without limitation, RV-7 at a spindle speed of 20 rpm, recognizing that the exact spindle may be selected as needed by one skilled in the art. Kinematic viscosity may be determined by dividing dynamic viscosity by the density of the liquid (at 25° C. and ambient conditions), as known in the art.

The Term Dv50 Spray particle refers to the average particle size of spray droplets and was done as per U.S. Patent Publication No. 2015/0000687. A Malvern Spraytec instrument was used to measure the particle size distribution following the manufacturer's instructions with test samples having a temperature between 20° C. to 22° C. and samples were sprayed perpendicular to the laser bean for 5 seconds. The lower the Dv50 the finer the aerosol. Finer aerosol indicates easy of atomization of the formula and vice versa, the higher the Dv50 the harder to atomize up to the point where no aerosol is formed and there will be no results for Dv50, or at least inconsistent spray quality.

The term surface tension refers to the tension of the surface film of a liquid caused by the attraction of the particles in the surface layer by the bulk of the liquid, which tends to minimize surface area. Surface tension measurements were done using Wilhelmy plate method ASTM D1331-20 method C, with a Krüss K100 tensiometer instrument used for all measurements.

The term light transmittance is the percent of incident light that pass thru a substance. Light transmittance may be measured using a spectrophotometer, such as, for example, a Spectronic Genesys 10 Vis Spectrophotometer available from Thermo Electron Corp (USA), wherein a light transmittance value greater than 80%, 85%, 90% or 95% at 25° C. indicates sufficient solubility in water.

### Gas Solubility Test Method

Gas solubility in a liquid was quantified using Henry's Law Constant, H, which is an equilibrium parameter with units of amount of solubilized gas per unit volume of liquid per unit pressure (e.g. mol/m3bar). Henry's Law Constant is related to the concentration of solubilized gas, C, and the equilibrium pressure, P, as C=HP. The Henry's Law Constant was quantified by measuring the transient pressure relaxation after adding gas to a known volume of liquid in a sealed pressure cell with a well-defined volume of 322 mL. The pressure relaxation was used to determine the number of moles of gas solubilized in a liquid volume at a given equilibrium pressure. The data was then plotted as the concentration of solubilized gas against the equilibrium pressure and fit to a line to extract the Henry's Law Constant from the slope.

The experimental setup for determining Henry's Law Constant includes a Parr 4848 Reactor Controller and temperature controlled T316 Stainless Steel pressure reaction apparatus (Model no. 4566). The apparatus includes a 300 mL jacketed vessel, digital and analog pressure gauges, a type J thermocouple, and a variable speed motor attached to an impeller. Transient pressure was recorded digitally at 5 second intervals using the SpecView32 software.

### Examples

Examples 57 and 58 are inventive examples for a single phase concentrate. Example 57 has about 27% emollient and Example 58 has about 38% emollient. In example 57 a 45 mm×162 mm can was filled with 100 g of concentrate composition and the can was pressurized to 130 psi with nitrogen. In example 58 a 45 mm×150 mm can was filled with 100 g of concentrate composition and the can was pressurized to 130 psi with nitrogen. In examples 59-62 a 45 mm×150 mm can was filled with 100 g of the respective concentrate composition and the can was pressurized to 100 psi with nitrogen.

Example 59 is an inventive example of an oil-in-water emulsion in the concentrate. Examples 60, 61, and 62 are inventive examples of water-in-oil emulsions in the concentrate.

| | Ex. 57 | Ex. 58 |
|---|---|---|
| Ethanol 200 proof | 64.65% | 52.79% |
| Dipropylene glycol | 18.78% | 37.67% |
| Caprilic/capric | 9.89% | |
| triglyceride | | |
| Water | 3.46% | 3.60% |
| Fragrance | 1.98% | 1.98% |
| Piroctone Olamine | 0.10% | 0.11% |
| Ethylhexyglycerin | | 0.99% |
| Trietyl citrate | | 0.99% |
| Citric acid | | 0.99% |
| Nitrogen | 1.15% | 0.88% |
| Viscosity (cP) | 7.50 | 11.00 |
| Surface Tension | 24.00 | 26.10 |
| (dyn/cm) | | |
| Viscosity/Surface | 0.31 | 0.42 |
| Tension | | |

| | Ex. 59 | Ex. 60 | Ex. 61 | Ex. 62 |
|---|---|---|---|---|
| Aluminum | 25.75% | 39.62% | 49.53% | 39.64% |
| Hydroxychloride 50% | | | | |
| Solution | | | | |
| Water | 56.46% | 9.91% | 9.91% | 9.91% |
| PPG-15 Stearyl Ether | 2.97% | | | |
| Steareth-2 | 1.88% | | | |
| Steareth-2 0 | 1.09% | | | |
| Propylene glycol | 9.91% | | | |
| Dipropylene Glycol | | 4.95% | 4.95% | 4.96% |
| PEG-9 | | 1.98% | 1.98% | 1.98% |
| Polydimethylsiloxyethyl | | | | |
| Dime | | | | |
| Isopropyl Myristate | | 4.95% | 4.95% | 31.22% |
| Cyclopentasiloxane | | 36.65% | 26.74% | 10.41% |
| Fragrance | 0.99% | 0.99% | 0.99% | 0.99% |
| Nitrogen | 0.95% | 0.95% | 0.95% | 0.89% |
| Concentrate Formula | 1200.00 | 43.75 | 242.30 | 50.00 |
| Viscosity (cP) | | | | |
| Continuous Phase | 7.25 | 6.25 | 6.50 | 10.00 |
| Viscosity (cP) | | | | |
| Surface Tension | 59.51 | 19.30 | 19.49 | 22.56 |
| (dyn/cm) | | | | |
| Viscosity/ | 0.12 | 0.32 | 0.33 | 0.44 |
| Surface Tension | | | | |

Examples 63-67 were made and Example 68 could be made as described herein. The examples were put or could be put into a pressurized can with the compressed gas.

| | Ex. 63 | Ex. 64 | Ex. 65 | Ex. 66 | Ex. 67 | Ex. 68 |
|---|---|---|---|---|---|---|
| | (wt. %) | (wt. %) | (wt. %) | (wt. %) | (wt. %) | (wt. %) |
| Aluminum | | | | | | 40.00 |
| Hydroxychloride 50% | | | | | | |
| Solution | | | | | | |
| Ethanol 200 proof | 67.24 | 48.27 | 64.89 | 61.42 | 63.51 | 5.00 |
| Piroctone Olamine | 0.12 | 0.12 | 0.12 | 0.12 | 0.11 | |
| Caprylic/Capric | 9.87 | | 9.76 | 2.85 | 9.33 | |
| Triglyceride-PrimG | | | | | | |
| Dipropylene Glycol | | 17.77 | 18.77 | 18.76 | | 20.00 |
| Triethyl Citrate | 17.78 | 3.95 | 2.96 | 2.96 | 16.79 | |
| Citric Acid | 1.78 | 0.40 | 0.30 | 0.30 | 1.68 | |
| Purified Water | | 26.23 | | 10.36 | | 28.00 |
| Fragrance | 1.98 | 1.97 | 1.98 | 1.98 | 1.87 | 2.00 |
| Compressed air | | | | 1.25 | | |
| Carbon Dioxide | | | | | 6.71 | 5.00 |
| Nitrogen | 1.24 | 1.29 | 1.23 | | | |

Examples 69-71 and 75 were made and Examples 72-74 and 76 could be made as described herein. The examples were put or could be put into a pressurized can with the compressed gas.

| | Ex. 69 | Ex. 70 | Ex. 71 | Ex. 72 | Ex. 73 | Ex. 74 | Ex. 75 | Ex. 76 |
|---|---|---|---|---|---|---|---|---|
| | (wt. %) | (wt. %) | (wt. %) | (wt. %) | (wt. %) | (wt. %) | (wt. %) | (wt. %) |
| Aluminum | 36.01 | 36.01 | 34.64 | | | | 40.00 | |
| Hydroxychloride 50% | | | | | | | | |
| Solution | | | | | | | | |
| Aluminum | | | | 38.00 | | 20.00 | | 40.00 |
| Sesquichloride 40% | | | | | | | | |
| solution | | | | | | | | |
| Aluminum | | | | | 35.00 | | | |
| Dichlorohydrate 28% | | | | | | | | |
| Solution | | | | | | | | |
| Water | 46.98 | 46.98 | 45.19 | 49.94 | 52.92 | 28.69 | 11.92 | 15.19 |
| Calcium Chloride | 1.00 | 1.00 | 0.96 | 1.00 | 1.00 | 0.52 | 1.02 | 1.01 |
| Glycine | 1.72 | 1.72 | 1.65 | 1.72 | 1.72 | 0.90 | 1.80 | 1.80 |
| PPG-15 Stearyl Ether | 3.00 | 3.00 | 2.89 | 3.01 | 3.01 | | | |
| Steareth-2 | 1.90 | 1.90 | 1.82 | 1.90 | 1.90 | | | |
| Steareth-20 | 1.10 | 1.10 | 1.05 | 1.10 | 1.10 | | | |
| Disodium EDTA | 0.10 | 0.10 | 0.10 | 0.10 | 0.10 | | | |
| Propylene glycol | | | | | | | | |
| Ethanol 200 proof | 4.94 | 4.94 | 4.56 | | | 5.00 | 5.00 | 10.00 |
| Dipropylene Glycol | | | | | | 10.00 | 4.96 | |
| PEG/PPG-18/18 | | | | | | 1.00 | | |
| Dimethicone | | | | | | | | |
| PEG-9 | | | | | | | 2.00 | 2.00 |
| Polydimethylsiloxyethyl | | | | | | | | |
| Dimethicone | | | | | | | | |
| Isopropyl myristate | | | | | | 4.95 | | 13.00 |
| Dimethicone 5 cst | | | | | | | | 12.00 |
| Cyclopentasiloxane | | | | | | 26.74 | 25.00 | |
| BHT | 0.05 | 0.05 | 0.05 | 0.05 | 0.05 | | 2.00 | 2.00 |
| Fragrance | 1.98 | 1.98 | 1.98 | 1.98 | 1.98 | 0.99 | 2.00 | 2.00 |
| Carbon Dioxide | | | 5.11 | | | | 4.30 | |
| Compressed air | | 1.23 | | | | | | |
| Nitrogen | 1.23 | | | 1.20 | 1.22 | 1.20 | | 1.00 |

### Combinations:

A. An aerosol personal care product comprising:
a. a pressurized container,
b. a compressed gas propellant contained within the container; and
c. a liquid concentrate contained within the container; wherein the liquid concentrate comprises at least 10%, by weight of the concentrate, of one or more emollients; and wherein the concentrate comprises a viscosity (cP) to surface tension (dyn/cm) ratio of at most 1, preferably at most 0.8, more preferably at most 0.5, an even more preferably at most 0.3.

B. The product according to paragraph A, wherein at least one emollient comprises a viscosity of at least 10 cP, preferably at least 20 cP according to the Viscosity Test Method described herein.

C. The product according to any preceding paragraph, wherein the liquid concentrate comprises a gas solubility at least 1e-4H (mol/m3 Pa) at 25° C., preferably at least 2e-4H (mol/m3 Pa) at 25° C., more preferably at least 3e-4H (mol/m3 Pa) at 25° C., and even more preferably at least at least 4e-4H (mol/m3 Pa) at 25° C. according to the Gas Solubility Test Method described herein.

D. The product according to any preceding paragraph, wherein the propellant is chosen from nitrogen, air, carbon dioxide, nitrous oxide, argon, helium, oxygen, or mixtures thereof.

E. The product according to any preceding paragraph, wherein the container further comprises a valve that produces atomization of the concentrate upon actuation and an actuator insert comprising an area; wherein the valve has a valve stem comprising a valve stem area.

F. The product according to paragraph E, wherein the atomization of the concentrate is effervescent atomization.

G. The product of paragraph E, comprising a ratio of the valve stem to the actuator insert area of less than 10, preferably less than 7, more preferably less than 5, and even more preferably less than 3.

H. The product according to any preceding paragraph, wherein the valve has a spray rate of at most 1 g/s, preferably from about 0.1 grams/sec to about 0.5 grams/sec, more preferably from about 0.2 grams/sec to about 0.4 grams/sec, and even more preferably from about 0.25 grams/sec to about 0.35 grams/sec.

I. The product according to any preceding paragraph, wherein the product is an antiperspirant comprising an antiperspirant active and/or deodorant product.

J. The product according to any preceding paragraph, wherein the liquid concentrate is a single-phase liquid concentrate.

K. The product of paragraph J, wherein the one or more emollients are chosen from propylene glycol, glycerol, polypropylene glycol, dipropylene glycol, tripropylene glycol, diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols, cyclopentasiloxane, dimethicone, mineral oils, petrolatum, water insoluble alkyl ethers, esters, carbonates, PPG-stearyl ether, isopropyl myristate and dipropyl heptyl carbonate, or mixtures thereof, preferably dipropylene glycol and/or capric/caprylic triglyceride.

L. The product according to paragraphs A-I, wherein the liquid concentrate is a water-in-oil emulsion comprising a continuous phase and a dispersed phase; wherein the continuous phase comprises the emollient; and wherein the emollient is water-insoluble.

M. The product of paragraph L, wherein the dispersed phase comprises water, water-soluble actives, and water-soluble emollients and wherein the emollient is chosen from cyclopentasiloxane, dimethicone, mineral oils, petrolatum, water insoluble alkyl ethers, esters, carbonates, PPG-15 stearyl ether, isopropyl myristate and dipropyl heptyl carbonate, or mixtures thereof.

N. The product according to paragraphs A-I, wherein the liquid concentrate is an oil-in-water emulsion comprising a continuous phase and a dispersed phase; wherein the continuous phase comprises the emollient; and wherein the emollient is water-soluble.

O. The product of paragraph N, wherein the water soluble emollients are chosen from propylene glycol, glycerol, polypropylene glycol, dipropylene glycol, tripropylene glycol, diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols, or mixtures thereof.

P. The product according to any preceding paragraph, wherein when sprayed the Dv50 is no more than 200 μm, alternatively no more than 175 μm, alternatively no more than 150 μm, alternatively no more than 115 μm.

Q. The product according to any preceding paragraph, wherein when sprayed the Dv50 is at least 50 μm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. All numeric values (e.g., dimensions, flow rates, pressures, concentrations, etc.) recited herein may be modified by the term “about”, even if not expressly so stated with the numeric value.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An aerosol personal care product comprising a composition, wherein the composition comprises:
a. a pressurized container;
b. a compressed gas propellant contained within the container, and
c. a liquid concentrate contained within the container; wherein the liquid concentrate comprises at least about 10%, by weight of the concentrate, of one or more emollients; and wherein the concentrate comprises a viscosity (cP) to surface tension (dyn/cm) ratio of at most about 1.

2. The product of claim 1, wherein the concentrate comprises a viscosity (cP) to surface tension (dyn/cm) ratio of at most about 0.8.

3. The product of claim 1, wherein at least one emollient comprises a viscosity of at least about 10 cP.

4. The product of claim 1, wherein at least one emollient comprises a viscosity of at least about cP.

5. The product of claim 1, wherein the liquid concentrate comprises a gas solubility at least 1e-4H (mol/m3 Pa) at 25° C.

6. The product of claim 1, wherein the propellant is chosen from nitrogen, air, carbon dioxide, nitrous oxide, argon, helium, oxygen, or mixtures thereof.

7. The product of claim 1, wherein the container further comprises a valve that produces atomization of the concentrate upon actuation and an actuator insert comprising an area; wherein the valve has a valve stem comprising a valve stem area.

8. The product of claim 7, wherein the atomization of the concentrate is effervescent atomization.

9. The product of claim 7, comprising a ratio of the valve stem to the actuator insert area of less than about 10.

10. The product of claim 1, wherein the product has an initial pressure of at least about 2 bars.

11. The product of claim 1, wherein the valve has a spray rate of at most about 1 g/s.

12. The product of claim 1, wherein the product is an antiperspirant comprising an antiperspirant active and/or deodorant product.

13. The product of claim 1, wherein the liquid concentrate is a single-phase liquid concentrate.

14. The product of claim 13, wherein the one or more emollients are chosen from propylene glycol, glycerol, polypropylene glycol, dipropylene glycol, tripropylene glycol, diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols, cyclopentasiloxane, dimethicone, mineral oils, petrolatum, water insoluble alkyl ethers, esters, carbonates, PPG-15 stearyl ether, isopropyl myristate and dipropyl heptyl carbonate, or mixtures thereof.

15. The product of claim 14, wherein the one or more emollients comprise dipropylene glycol and/or capric/caprylic triglyceride.

16. The product of claim 1, wherein the liquid concentrate is a water-in-oil emulsion comprising a continuous phase and a dispersed phase; wherein the continuous phase comprises the emollient; and wherein the emollient is water-insoluble.

17. The product of claim 16, wherein the dispersed phase comprises water, water-soluble actives, and water-soluble emollients.

18. The product of claim 16, wherein the emollient is chosen from cyclopentasiloxane, dimethicone, mineral oils, petrolatum, water insoluble alkyl ethers, esters, carbonates, PPG-15 stearyl ether, isopropyl myristate and dipropyl heptyl carbonate, or mixtures thereof.

19. The product of claim 1, wherein the liquid concentrate is an oil-in-water emulsion comprising a continuous phase and a dispersed phase; wherein the continuous phase comprises the emollient; and wherein the emollient is water-soluble.

20. The product of claim 19, wherein the water soluble emollients are chosen from propylene glycol, glycerol, polypropylene glycol, dipropylene glycol, tripropylene glycol, diethylene glycol, triethylene glycol, PEG-4, PEG-8, 1,2 pentanediol, 1,2 hexanediol, hexylene glycol, glycerin, C2 to C20 monohydric alcohols, C2 to C40 dihydric or polyhydric alcohols, water soluble alkyl ethers of polyhydric and monohydric alcohols, or mixtures thereof.
